(19) United States
(12) Patent Application Publication
(10) Pub. No.: US 2026/0014013 A1
(43) Pub. Date: Jan. 15, 2026

(54) POSTURE CORRECTION DEVICE

(71) Applicant: Kenichi TANAKA, Tokyo (JP)

(72) Inventor: Kenichi TANAKA, Tokyo (JP)

(21) Appl. No.: 18/837,730

(22) PCT Filed: Feb. 10, 2023
(86) PCT No.: PCT/JP2023/004582

§ 371 Date: Mar. 3, 2025

(30) Foreign Application Priority Data
Feb. 14, 2022 (JP) 2022-020255

(51) Int. Cl. A61F 5/02

(52) U.S. Cl. CPC A61F 5/028

(57) ABSTRACT

To provide a user-friendly instrument capable of easily performing motions of rehabilitation or the like and daily activities even in a worn state. A jaw receiving portion and a support portion are included. The jaw receiving portion includes a jaw receiving portion main body at one end thereof. The support portion includes a support portion main body, a shoulder portion, and a wearing portion. The support portion main body is configured to be able to support the jaw receiving portion. The shoulder portion is configured to be able to suspend the support portion main body from a shoulder of a user. The wearing portion is configured to be able to fix the support portion main body to a body of the user.

### TECHNICAL FIELD

The present invention relates to a posture correction instrument for improving a symptom of “neck lowering”.

### BACKGROUND ART

Neck lowering refers to a state in which a muscle group around a neck cannot support the weight of a head and the neck naturally lowers forward, and a state in which it is difficult to raise the lowered neck.

Neck lowering is caused by muscle weakness, excessive tension of muscles, and the like, and can occur due to diseases related to muscles, such as Parkinson's disease, amyotrophic lateral sclerosis (ALS), dystonia, and myopathy, or simple aging.

In particular, symptoms that develop in patients with Parkinson's disease are representative, and according to the National Center for Neurology and Psychiatry, 24,000 patients, which is about 16% of about 150,000 patients with Parkinson's disease in Japan, suffer from neck lowering.

In addition, in view of aging of the society, it can be easily inferred that there are a very large number of potential people who cannot maintain a correct posture due to lowering of their jaws due to a decrease in muscle strength around a neck, a waist bend due to aging, a deviation between the neck and a head due to scoliosis, a hunched back, or the like.

For this situation, various instruments and orthoses for fixedly holding the position of a neck and improving a posture have been devised.

For example, Patent Literature 1 describes a cervical orthosis capable of enhancing a wearing feeling, fixability, and the like by making a lower jaw support and an occipital region support freely adjustable not only in height but also in angle in forwardly and backwardly bending directions, and adjusting the forwardly and backwardly bending positions of the cervical vertebra according to various symptoms.

In addition, Patent Literature 2 discloses a neck manipulative instrument that allows a user to hold a correct posture by placing a jaw on the instrument to support the weight of a head, and can correct the position of a cervical bone of the user by pushing up the jaw.

### CITATION LIST

### Patent Literature

Patent Literature 1: JP 2000-14686 A
Patent Literature 2: JP 2011-50626 A

### SUMMARY OF INVENTION

### Technical Problem

However, the inventions described in Patent Literatures 1 and 2 are not mainly aimed at correcting neck lowering, and it is difficult to perform rehabilitation around a neck while a user wears each of the instruments of the inventions.

In particular, in treatment of Parkinson's disease and the like, it is desired to sufficiently enhance an effect of medicine by performing not only medication treatment but also appropriate rehabilitation according to a disease state. Therefore, emphasis should be placed on correction of a posture, and an instrument that is easily used during rehabilitation should be used.

The present invention has been made in view of the above problems, and it is an object of the present invention to provide a user-friendly instrument capable of easily performing motions of rehabilitation or the like and daily activities even in a worn state.

### Solution to Problem

In order to solve the above problems, the present invention includes
a jaw receiving portion and a support portion, in which
the jaw receiving portion includes a jaw receiving portion main body at one end thereof,
the support portion includes a support portion main body, a shoulder portion, and a wearing portion,
the support portion main body is configured to be able to support the jaw receiving portion,
the shoulder portion is configured to be able to suspend the support portion main body from a shoulder of a user, and
the wearing portion is configured to be able to fix the support portion main body to a body of the user.

According to the present invention, since the jaw receiving portion supports a jaw of a user, a movable range of a neck can be made wide, and at the same time, movement of the jaw receiving portion can be suppressed by fixing the support portion main body that supports the jaw receiving portion to a body of the user by the wearing portion. Therefore, the jaw can be supported with both stability around the neck and the degree of freedom even in a moving scene of rehabilitation, daily life, or the like.

In a preferred mode of the present invention, the jaw receiving portion is configured such that the jaw receiving portion main body is capable of spherical movement.

With such a configuration, a user can perform motions such as shaking a head, tilting, and turning around, and at the same time, a direction of action of a push-up force can be adjusted by adjusting a spherical position of the jaw receiving portion main body with respect to the jaw receiving portion. Therefore, it is possible to cope with neck lowering accompanied by a body torsion.

In a preferred mode of the present invention, the jaw receiving portion is a substantially rod-shaped body that is stretchable.

With such a configuration, a user can adjust the jaw receiving portion to an optimum length for the user, and at the same time, durability is improved as compared with a conventional product which is often formed of a plate-shaped body.

In a preferred mode of the present invention, the jaw receiving portion further includes a tabletop fixing portion that detachably fixes the jaw receiving portion to an arbitrary tabletop.

With such a configuration, a user can fix the jaw receiving portion to a tabletop of a business work desk, a dining table, a work desk, or the like, and can use the present instrument even during business work, eating, or other work. Therefore, it is possible to reduce time of a neck lowering state and to largely contribute to correcting a posture of the user.

In a preferred mode of the present invention, the jaw receiving portion is configured such that the jaw receiving portion and the tabletop fixing portion are detachable.

With such a configuration, a user can selectively use the present instrument in a use mode of wearing the present instrument on a body or fixing the present instrument to a tabletop. Therefore, a use scene of the present instrument is increased, and time of a neck lowering state is reduced, which further largely contributes to posture correction of the user.

In a preferred mode of the present invention, the support portion is configured to be able to adjust a horizontal position of the support portion main body.

With such a configuration, a user can adjust a horizontal position of the support portion main body to adjust a horizontal component of a push-up force to the jaw. Therefore, it is possible to cope with neck lowering accompanied by a body torsion by adjusting a direction of the push-up force to be applied.

In a preferred mode of the present invention, the support portion main body is a bag body.

With such a configuration, the support portion main body supports the jaw receiving portion in a non-fixed manner. Therefore, the support portion main body can increase the degree of freedom of movement of a user, and at the same time, can house the components therein when the present instrument (X) is not used.

In a preferred mode of the present invention, the shoulder portion includes a pressing portion, the jaw receiving portion is a rod-shaped body that pushes up and supports a jaw of a user, the support portion main body supports the jaw receiving portion from below, the shoulder portion suspends and supports the support portion main body, and the pressing portion presses a back of the user by the weight of a head of the user applied to the shoulder portion via the jaw receiving portion and the support portion main body.

With such a configuration, muscles of a breast of a user are expanded, a winding shoulder of the user is corrected, and a force is applied so as to erect a head of the user. As a result, the user can more easily support his or her neck.

In a preferred mode of the present invention, a spring is disposed in the support portion main body, and the spring supports a lower end of the jaw receiving portion.

With such a configuration, even when a body moves up and down with the instrument attached, for example, when a user walks, it is possible to suppress a situation in which the jaw receiving portion is detached.

In a preferred mode of the present invention, a cylindrical body is disposed in the support portion main body, and the spring is disposed in the cylindrical body.

With such a configuration, the cylindrical body can further suppress a situation in which the jaw receiving portion is detached while preventing deviation of the jaw receiving portion in a horizontal direction.

In a preferred mode of the present invention, a cane attachment is further included, the cane attachment includes a multi-legged attachment and a grip attachment, the multi-legged attachment is detachably attached to a lower end of the stretchable rod-shaped body, and the grip attachment is detachably attached to an upper end of the stretchable rod-shaped body.

With such a configuration, a user can use the present correction instrument like a cane as necessary.

### Advantageous Effects of Invention

According to the present invention, it is possible to provide a user-friendly instrument capable of easily performing motions of rehabilitation or the like and daily activities even in a worn state.

### BRIEF DESCRIPTION OF DRAWINGS

FIG. 1 is a schematic view of a posture correction instrument according to an embodiment of the present invention.

FIG. 2 illustrates a schematic view and an exploded view of a jaw receiving portion according to the embodiment of the present invention.

FIG. 3 is a schematic view of a support portion according to the embodiment of the present invention.

FIG. 4 is a schematic view of a torsion adjusting portion according to the embodiment of the present invention.

FIG. 5 is a use example of a posture correction instrument according to the embodiment of the present invention.

FIG. 6 is a use example of the posture correction instrument according to the embodiment of the present invention.

FIG. 7 is a use example of the posture correction instrument according to the embodiment of the present invention.

FIG. 8 is a use example of a jaw receiving portion according to the embodiment of the present invention.

FIG. 9 is a schematic view of the jaw receiving portion according to the embodiment of the present invention.

FIG. 10 is a schematic view of a support portion main body according to the embodiment of the present invention.

FIG. 11 is a schematic view of a shoulder wearing portion according to the embodiment of the present invention.

FIG. 12 is a use example of a cane attachment according to the embodiment of the present invention.

### DESCRIPTION OF EMBODIMENT

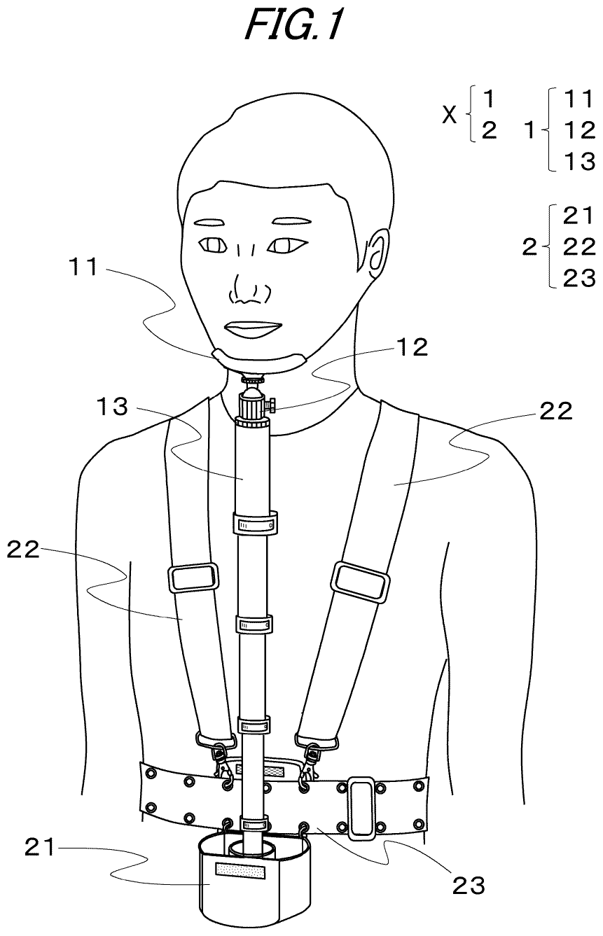

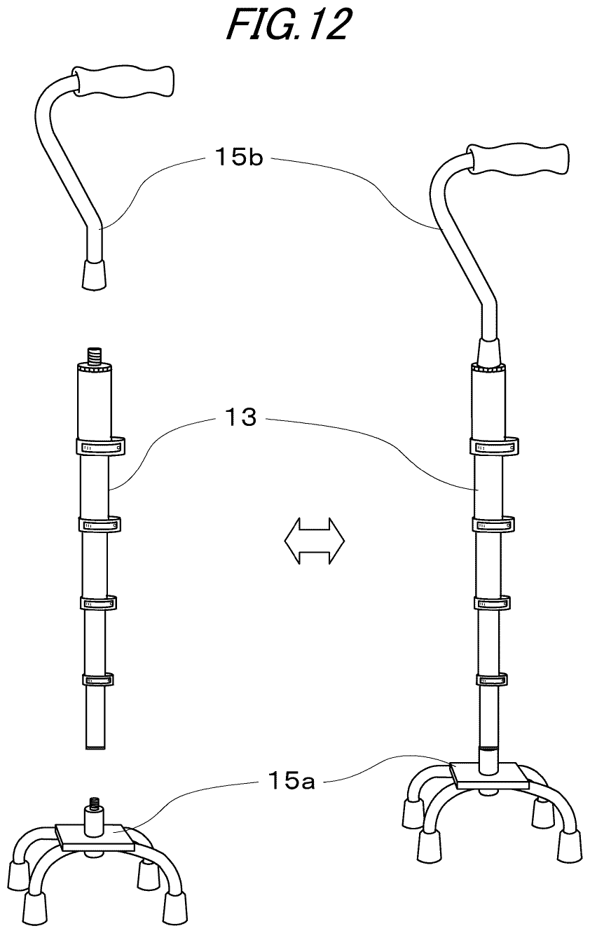

Hereinafter, a posture correction instrument according to an embodiment of the present invention will be described with reference to FIGS. 1 to 12. Note that the following embodiment is an example of the present invention, and the present invention is not limited to the following embodiment. A reference sign (X) indicates the posture correction instrument according to the present embodiment.

As illustrated in FIG. 1, the posture correction instrument (X) includes a jaw receiving portion (1) and a support portion (2).

The jaw receiving portion (1) includes a jaw receiving portion main body (11), a connecting portion (12), and a stretchable portion (13), and the support portion (2) includes a support portion main body (21), a shoulder portion (22), and a wearing portion (23).

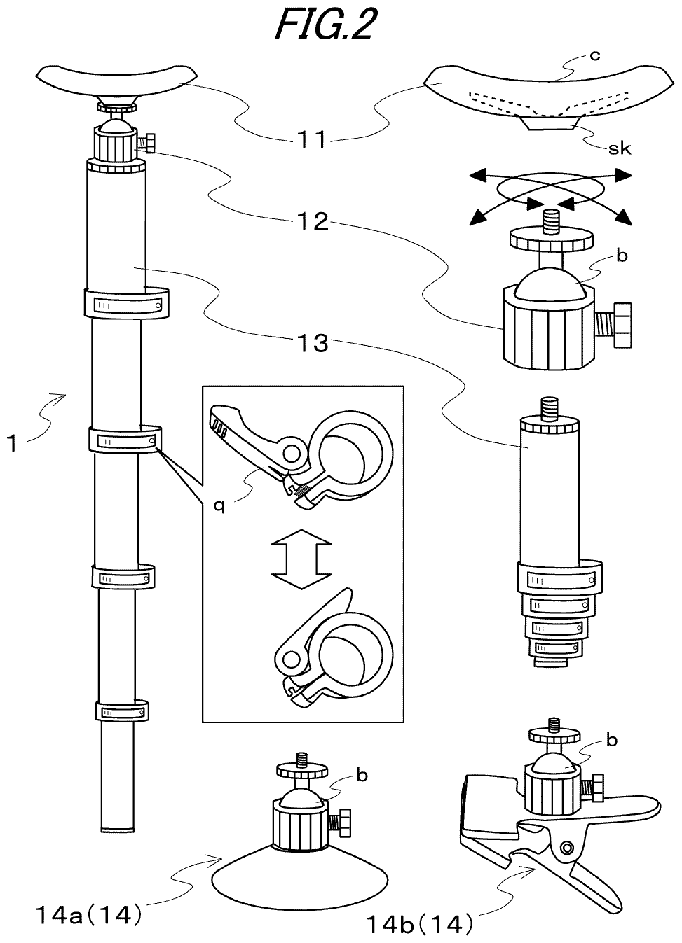

As illustrated in FIG. 2, the jaw receiving portion (1) includes the jaw receiving portion main body (11), the connecting portion (12), and the stretchable portion (13), each of which is detachably configured by a screw.

The jaw receiving portion main body (11) includes a contact portion (c) and a skeleton portion (sk). Note that the contact portion (c) is a portion on which a jaw of a user is directly placed, and therefore is preferably formed of a material having a good touch feeling in consideration of flexibility, air permeability, and the like, and preferably has a recess that fits the jaw.

Specifically, the contact portion (c) can be formed of a soft material such as silicone rubber.

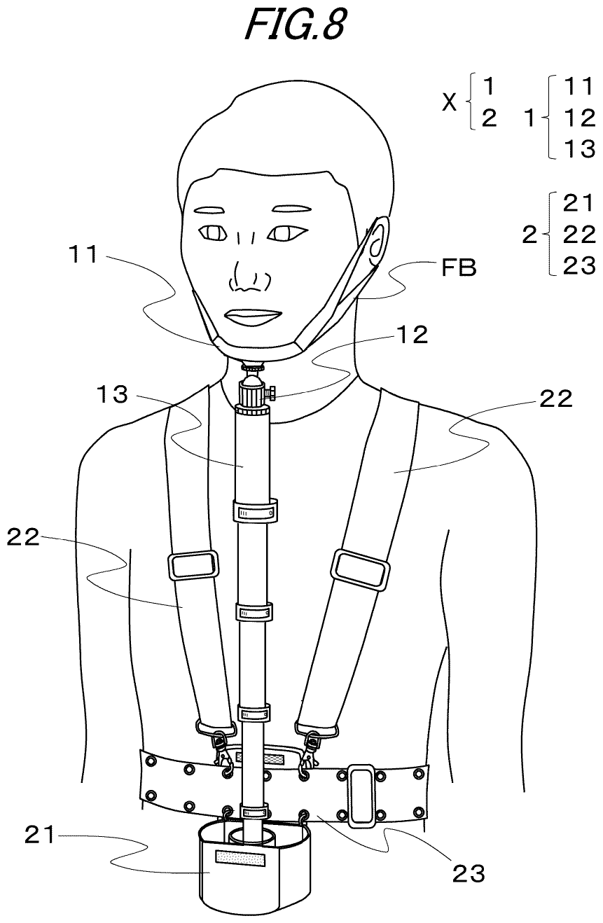

In addition, the jaw receiving portion main body (11) may include a fixing band (FB) that can be hung on an ear or an occipital region (FIG. 8). The fixing band (FB) prevents the jaw receiving portion main body (11) from being detached from a jaw of a user.

The connecting portion (12) includes a spherical joint (b) at a part where the connecting portion (12) is connected to the jaw receiving portion main body (11), and is configured to be capable of spherical motion of the connected jaw receiving portion main body (11). As a result, a user can perform motions such as shaking a head, tilting, and turning around without any problem even when wearing the instrument.

In addition, the spherical joint (b) can be prevented from moving by tightening a lateral screw. Therefore, it is also easy to fix a spherical position of the jaw receiving portion main body (11).

Note that the spherical joint b may be a fitting type joint that is not fixed by a screw.

The stretchable portion (13) can be expanded and contracted by opening and closing a lever (q) disposed at a predetermined position. Specifically, the stretchable portion (13) is expanded or contracted to an arbitrary length in a state where the lever (q) is opened, and then the lever (q) is closed to fix the length.

Note that, here, a lever system that is widely used as a stretchable mechanism such as a tripod for a camera has been described as an example, but any other configuration may be used as long as the configuration can be expanded and contracted.

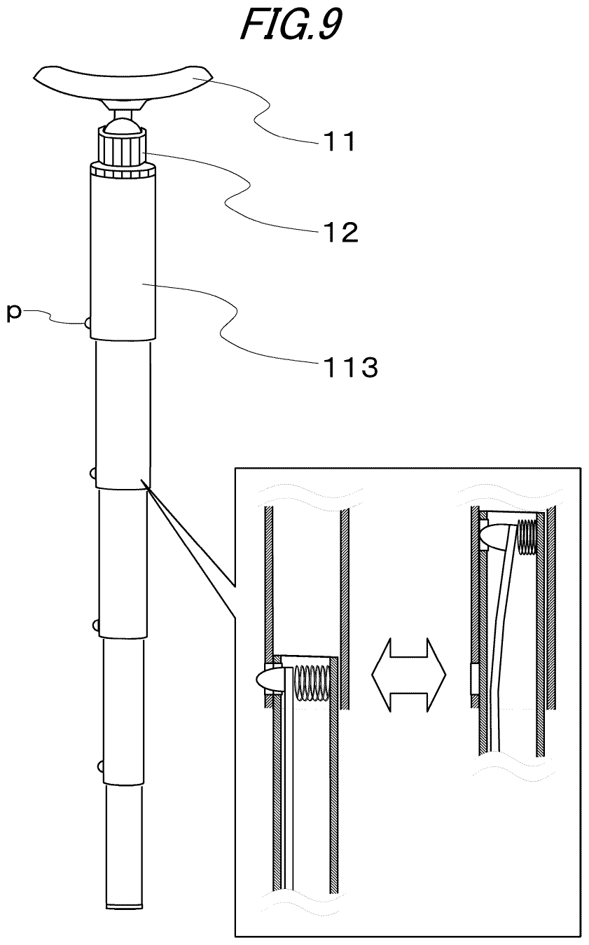

For example, as illustrated in FIG. 9, the stretchable portion (13) may have a configuration like a stretchable portion (113).

The stretchable portion (113) can be contracted by pushing in a pin (p), and the pin is fitted into a hole by pulling up and down the stretchable portion (113) to maintain an expanded state.

Note that a bone conduction type speaker (not illustrated) may be incorporated in a hollow portion of the stretchable portions (13), (113), and the like. The bone conduction type speaker can notify a user of, for example, time of exercise or medication.

The jaw receiving portion (1) includes a tabletop fixing base (14a) and a tabletop fixing clip (14b) as the tabletop fixing portion (14). The tabletop fixing portion (14) is detachably attached to an end of the stretchable portion (13) opposite to a side where the jaw receiving portion main body 11 is attached.

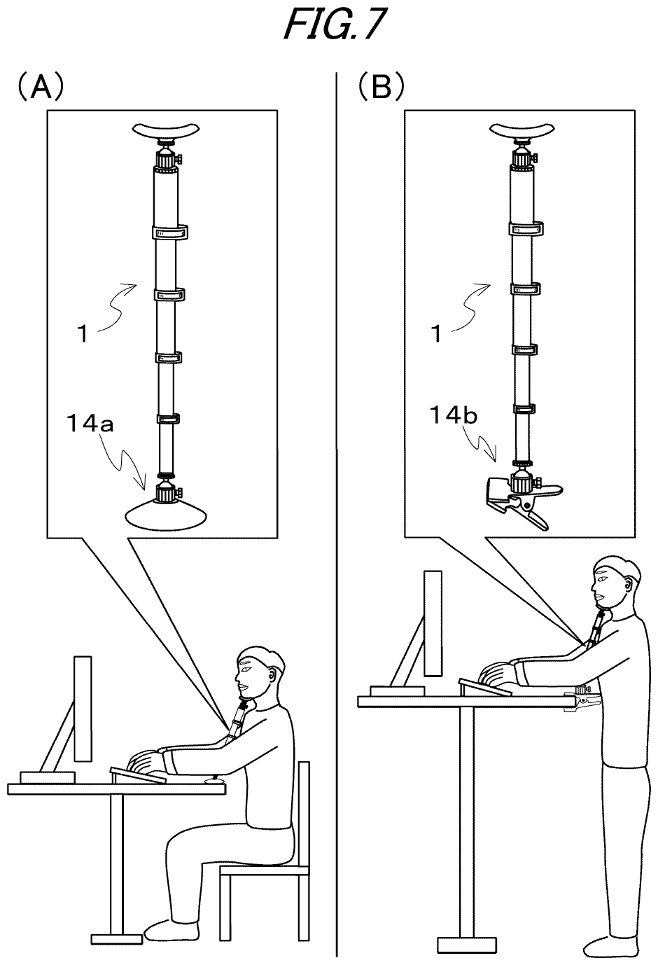

In addition, the tabletop fixing base (14a) and the tabletop fixing clip (14b) each include a spherical joint (b), and the jaw receiving portion (1) is also capable of spherical movement at one end of the spherical joint (b). The tabletop fixing base (14a) and the tabletop fixing clip (14b) allow the present instrument (X) to be suitably used also during work. Details of a use method in this case will be described later with reference to FIG. 7.

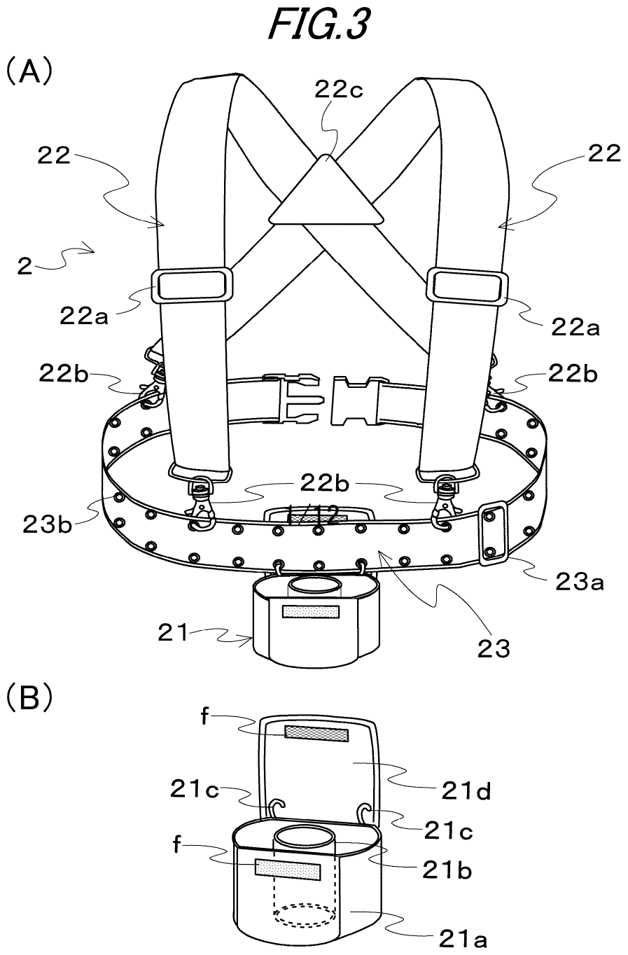

As illustrated in FIG. 3(A), the support portion (2) includes the support portion main body (21), the shoulder portion (22), and the wearing portion (23), which are detachably configured.

As illustrated in FIG. 3(B), the support portion main body (21) includes a bag body (21a), a cylindrical body (21b), an attachment portion (21c), and lid portion (21d).

By inserting the jaw receiving portion (1) into the bag body (21a), the jaw receiving portion can be supported in a non-fixed manner. In addition, the cylindrical body (21b) smaller than the bag body (21a) is included in the bag body (21a), and the jaw receiving portion (1) can be thereby inserted into the cylindrical body (21b) and can be supported more stably.

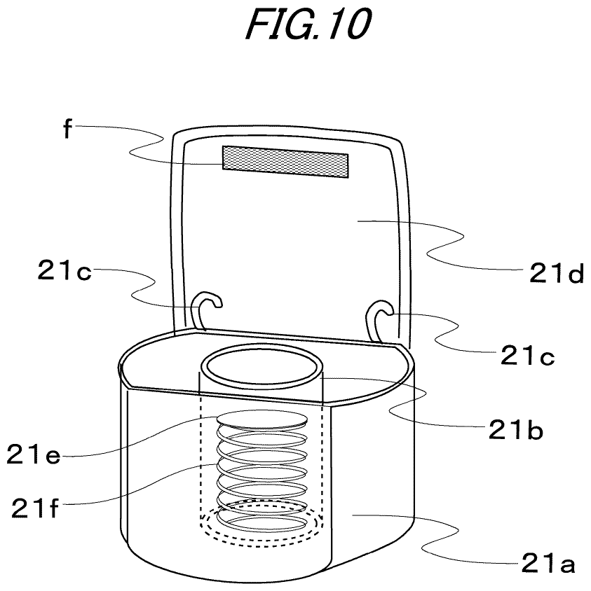

Note that the bag body (21a) or the cylindrical body (21b) may include a spring (21f) having a support base (21e) therein (FIG. 10). The support base (21e) receives a lower end of the jaw receiving portion (1) with a recess or the like. The support base (21e) may be detachably attached to the lower end of the jaw receiving portion (1) with a magnet, a screw, or the like.

The spring (21f) pushes up the lower end of the jaw receiving portion (1) to suppress a situation in which the jaw receiving portion main body (11) is detached from a jaw of a user.

The attachment portion (21c) can be detachably attached to an attachment hole (23b) of the wearing portion (23) described later. Note that although a hook is illustrated here, any member may be used as long as the member can be attached to the attachment hole 23b, such as a carabiner or a loop clutch.

The lid portion (21d) can freely open and close the bag body (21a) with a hook-and-loop fastener (f). As a result, a user can store the components in the bag body 21a when not wearing the present instrument (X).

As illustrated in FIG. 3(A), the shoulder portion (22) includes a length adjusting portion (22a), an attachment portion (22b), and a pressing portion (22c).

The length adjusting portion (22a) can adjust the length of a belt of the shoulder portion (22). Therefore, a user can wear the shoulder portion (22) with a length that matches his or her body.

The attachment portion (22b) can be detachably attached to the attachment hole (23b) of the wearing portion (23) described later. Note that although a loop clutch is illustrated here, any member may be used as long as the member can be attached to the attachment hole (23b), such as a carabiner or a hook.

The pressing portion (22c) presses a back of a user, particularly a portion between left and right shoulder blades, toward a ventral side of the user when the user wears the present instrument (X). As a result, muscles of a breast of the user are expanded, and a force to erect a head thereby acts.

Note that the pressing portion (22c) is a member that is strongly pressed against a body of a user, and therefore preferably has certain flexibility.

In addition, the pressing portion (22c) preferably has a triangular shape so as to fit a portion between right and left shoulder blades.

As illustrated in FIG. 3(A), the wearing portion (23) includes a length adjusting portion (23a) and the attachment hole (23b).

The length adjusting portion (23a) can adjust the length of a belt of the wearing portion (23). Therefore, a user can wear the wearing portion (23) with a length that matches his or her body.

A plurality of the attachment holes (23b) are formed in a substantially horizontal direction of the wearing portion (23) at predetermined intervals such that the attachment holes (23b) are arranged vertically in two rows, and the above-described attachment portions (21c) and (22b) can be detachably attached to the two rows of the attachment holes (23b), respectively.

As a result, a user can adjust horizontal attachment positions of the support portion main body (21) and the shoulder portion (22) according to his or her physical features.

That is, a user can adjust a horizontal component of a push-up force to a jaw by adjusting a horizontal position of the support portion main body (21) that supports the jaw receiving portion (1).

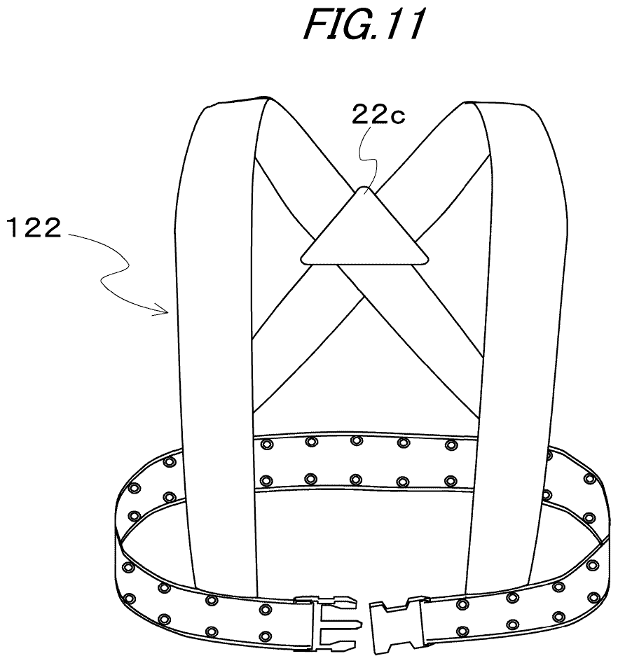

Note that, as illustrated in FIG. 11, the shoulder portion (22) and the wearing portion (23) may be integrally formed into a shoulder wearing portion (122). The entire shoulder wearing portion (122) is integrally formed and a buckle of a belt is on a ventral side of a user, and is therefore easily attached and detached.

The support portion (2) may further include a torsion adjusting portion (24).

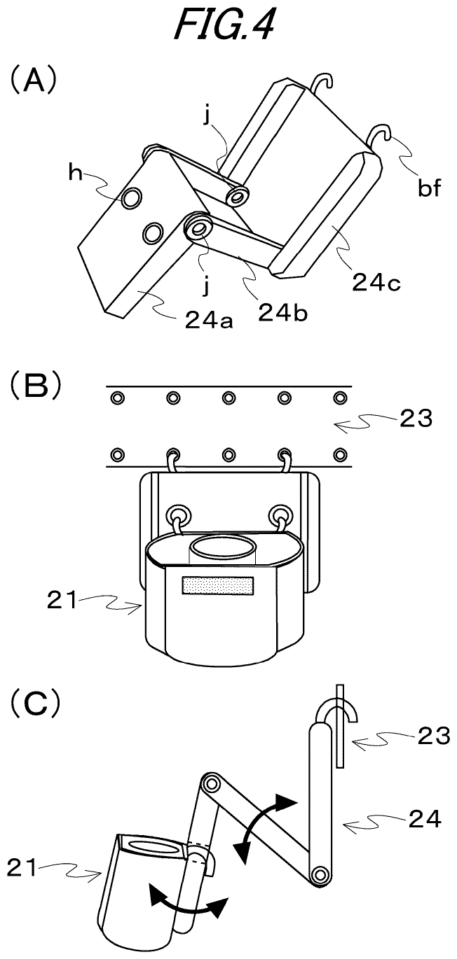

As illustrated in FIG. 4(A), the torsion adjusting portion (24) includes a first adjusting body (24a), a connecting portion (24b), and a second adjusting body (24c).

The first adjusting body (24a) has a support portion main body attachment hole h, and the attachment portion (21c) of the support portion main body (21) can be detachably attached to the support portion main body attachment hole h.

The connecting portion (24b) connects the first adjusting body (24a) and the second adjusting body (24c) via a joint portion (j). Therefore, the first adjusting body (24a), the connecting portion (24b), and the second adjusting body (24c) are configured to be rotatable about the joint portion (j).

The second adjusting body (24c) has a wearing portion attachment portion (bf), and the wearing portion attachment portion (bf) can be detachably attached to the attachment hole (23b) of the wearing portion (23).

With the above configuration, as illustrated in FIG. 4(B), the torsion adjusting portion (24) can be detachably incorporated between the wearing portion (23) and the support portion main body (21).

In addition, as illustrated in FIG. 4(C), the torsion adjusting portion (24) is incorporated between the wearing portion (23) and the support portion main body (21), whereby each part of the torsion adjusting portion (24) can be rotated to adjust a front-back position of the support portion main body (21) with respect to the wearing portion (23).

That is, a user can adjust a front-back component of a push-up force to a jaw by adjusting a front-back position of the support portion main body (21) that supports the jaw receiving portion (1).

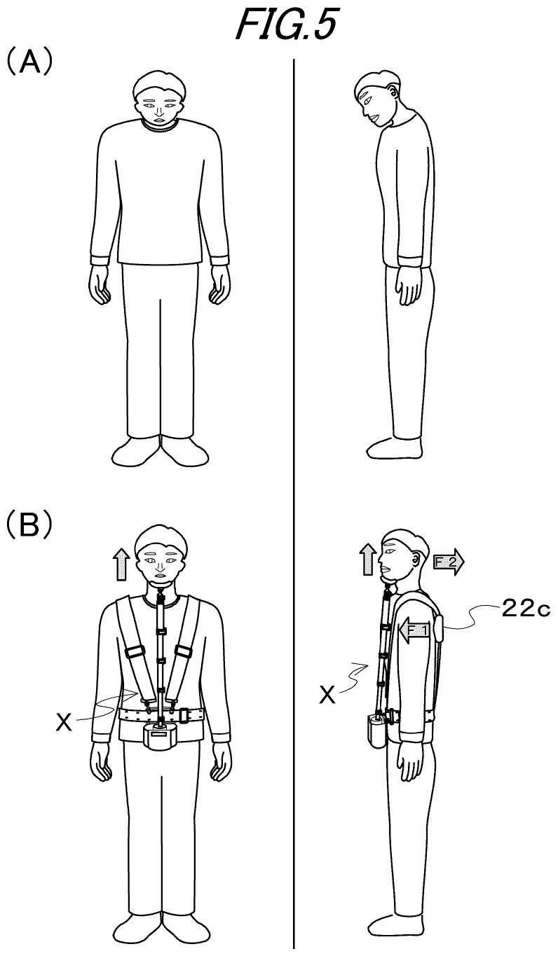

FIGS. 5 to 7 illustrate examples of a use mode of the present instrument (X).

As illustrated in FIG. 5(A), a patient having a symptom of neck lowering naturally has a neck lowered forward, and therefore his or her daily life is difficult.

Therefore, as illustrated in FIG. 5(B), a user can easily support his or her neck by wearing the present instrument (X).

In addition, when the present instrument (X) is worn, the jaw receiving portion (1) pushes up and supports a jaw of the user, whereby the weight of a head of the user is applied to the support portion main body (21) that supports the jaw receiving portion (1) and the shoulder portion (22) that supports the support portion main body (21). With this weight, the wearing portion (23) and the shoulder portion (22) are pulled downward, and the pressing portion (22c) is pressed against a back (between left and right shoulder blades) of the user. As a result, the pressing portion (22c) presses (F1) the back of the user toward a ventral side, and muscles of a breast of the user are expanded. Then, a winding shoulder is corrected and a force (F2) acts to erect the head. Therefore, the user can support his or her neck more easily.

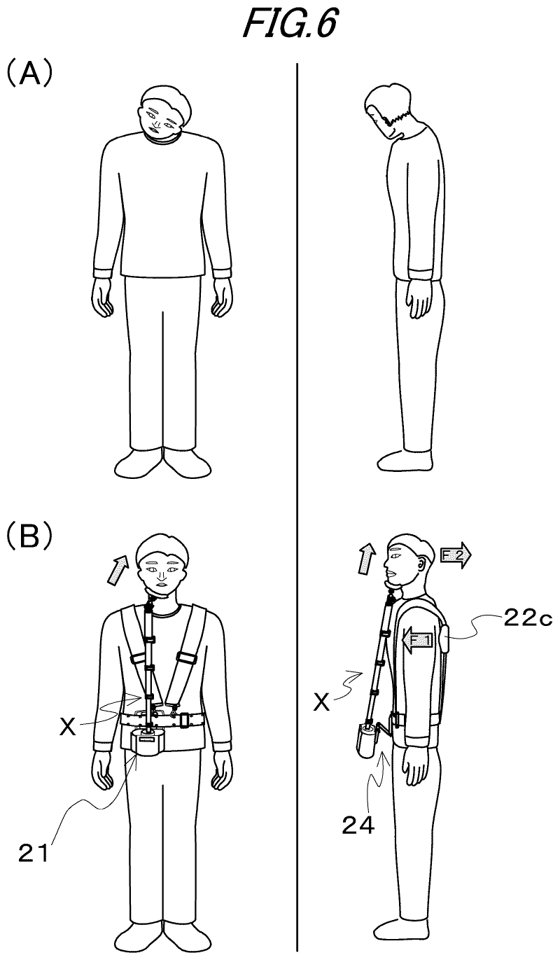

FIG. 6 illustrates an example of a use mode of the present instrument (X).

As illustrated in FIG. 6(A), a patient having a symptom of neck lowering accompanied by a body torsion may naturally have a neck lowered forward and a head turned sideways.

Therefore, as illustrated in FIG. 6(B), by wearing the present instrument (X) in which a horizontal position of the support portion main body (21) is adjusted, a user can support his or her neck at a normal position.

In addition, the user can adjust a front-back position of the support portion main body (21) by the torsion adjusting portion (24), and therefore can adjust a front-back direction of a push-up force to the jaw according to a use feeling and a wearing feeling.

As illustrated in FIGS. 5 and 6, since the entire present instrument (X) is worn on a body of a user and does not narrow a neck or other movable ranges, the user can easily perform motions of rehabilitation or the like and daily activities even in a worn state.

As illustrated in FIG. 7, the jaw receiving portion (1) can be used alone by attaching the tabletop fixing portion (14) to an end thereof.

For example, FIG. 7(A) illustrates an example in which the jaw receiving portion (1) to which the tabletop fixing base (14a) is attached is fixed onto a workbench and work is performed in a sitting position.

For example, FIG. 7(B) illustrates an example in which the jaw receiving portion (1) to which the tabletop fixing clip (14b) is attached is fixed onto a workbench and work is performed in a standing position.

Note that a surface having a large coefficient of friction, a sucker, or the like is disposed on a ground contact surface of the tabletop fixing base (14a), whereby the tabletop fixing base (14a) can be detachably fixed to a tabletop.

By using the present instrument (X) in this manner, a user of the present instrument (X) can select an easy posture according to stiffness of his or her body while reducing time of a neck lowering state as much as possible even during work such as business work or housework, and therefore can approach a correct posture.

Note that, in the present embodiment, the tabletop fixing base (14a) and the tabletop fixing clip (14b) have been described as the tabletop fixing portion (14). However, the tabletop fixing portion (14) may have any configuration as long as it can be detachably fixed to a tabletop.

In addition, the present instrument (X) may include a cane attachment (15) (FIG. 15).

The cane attachment (15) includes a multi-legged attachment (15a) and a grip attachment (15b).

As illustrated in FIG. 15, the multi-legged attachment (15a), the stretchable portion (13), and the grip attachment (15b) can be assembled like a cane. As a result, a user can use the stretchable portion (13) like a cane.

According to the present invention, while the jaw receiving portion (1) supports a jaw of a user, a movable range of a neck can be made wide, and at the same time, movement of the jaw receiving portion (1) can be suppressed by fixing the support portion main body (21) that supports the jaw receiving portion (1) to a body of the user by the wearing portion (23). Therefore, the jaw can be supported with both stability around the neck and the degree of freedom even in a moving scene.

In addition, the jaw receiving portion (1) is configured such that the jaw receiving portion main body (11) is capable of spherical movement. Therefore, a user can perform motions such as shaking a head, tilting, and turning around, and at the same time, a direction of action of a push-up force can be adjusted by adjusting a spherical position of the jaw receiving portion main body (11) with respect to the jaw receiving portion (1). Therefore, it is possible to cope with neck lowering accompanied by a body torsion.

In addition, the jaw receiving portion (1) is a substantially rod-shaped body that is stretchable. Therefore, a user can adjust the jaw receiving portion (1) to an optimum length for the user, and at the same time, durability is improved as compared with a conventional product that is often formed of a plate-shaped body.

In addition, the jaw receiving portion (1) further includes the tabletop fixing portion (14) that detachably fixes the jaw receiving portion (1) to an arbitrary tabletop. Therefore, a user can fix the jaw receiving portion (1) to a tabletop of a work desk or the like, can use the present instrument (X) even during work, can reduce time of a neck lowering state, and can largely contribute to a posture correction of the user.

In addition, the jaw receiving portion (1) is configured such that the jaw receiving portion (1) and the tabletop fixing portion (14) are detachable. Therefore, a user can selectively use the present instrument (X) in wearing the present instrument (X) on a body or fixing the present instrument (X) to a tabletop. Therefore, a use scene of the present instrument is increased, and time of a neck lowering state is reduced, which further largely contributes to posture correction of the user.

In addition, the support portion (2) is configured to be able to adjust a horizontal position of the support portion main body (21). Therefore, a user can adjust a horizontal position of the support portion main body (21) to adjust a horizontal component of a push-up force to a jaw. Therefore, it is possible to cope with neck lowering accompanied by a body torsion by adjusting a direction of the push-up force to be applied.

In addition, the support portion main body (21) is a bag body. Therefore, the support portion main body (21) supports the jaw receiving portion (1) in a non-fixed manner, therefore, can increase the degree of freedom of movement of a user, and at the same time, can house the components therein when the present instrument (X) is not used.

Note that the components and functions described in the above-described embodiment are merely examples, and various modifications can be made on the basis of design requirements and the like.

### REFERENCE SIGNS LIST

X Posture correction instrument
1 Jaw receiving portion
11 Jaw receiving portion main body
12 Connecting portion
13 Stretchable portion
14 Tabletop fixing portion
2 Support portion
21 Support portion main body
22 Shoulder portion
23 Wearing portion
24 Torsion adjusting portion

1. A posture correction instrument comprising
a jaw receiving portion and a support portion, wherein
the jaw receiving portion includes a jaw receiving portion main body at one end thereof,
the support portion includes a support portion main body, a shoulder portion, and a wearing portion,
the support portion main body is configured to be able to support the jaw receiving portion,
the shoulder portion is configured to be able to suspend the support portion main body from a shoulder of a user, and
the wearing portion is configured to be able to fix the support portion main body to a body of the user.

2. The posture correction instrument according to claim 1, wherein the jaw receiving portion is configured such that the jaw receiving portion main body is capable of spherical movement.

3. The posture correction instrument according to claim 1, wherein the jaw receiving portion is a stretchable rod-shaped body.

4. The posture correction instrument according to claim 1, wherein the jaw receiving portion further includes a tabletop fixing portion that detachably fixes the jaw receiving portion to an arbitrary tabletop.

5. The posture correction instrument according to claim 4, wherein the jaw receiving portion is configured such that the jaw receiving portion and the tabletop fixing portion are detachable.

6. The posture correction instrument according to claim 1, wherein the support portion is configured to be able to adjust a horizontal position of the support portion main body.

7. The posture correction instrument according to claim 1, wherein the support portion main body is a bag body.

8. The posture correction instrument according to claim 7, wherein a cylindrical body is disposed in the support portion main body.

9. The posture correction instrument according to claim 1, wherein
the shoulder portion includes a pressing portion,
the jaw receiving portion is a rod-shaped body that pushes up and supports a jaw of a user,
the support portion main body supports the jaw receiving portion from below,
the shoulder portion suspends and supports the support portion main body, and
the pressing portion presses a back of the user by a weight of a head of the user applied to the shoulder portion via the jaw receiving portion and the support portion main body.

10. The posture correction instrument according to claim 7, wherein
a spring is disposed in the support portion main body, and
the spring supports a lower end of the jaw receiving portion.

11. The posture correction instrument according to claim 10, wherein
a cylindrical body is disposed in the support portion main body, and
the spring is disposed in the cylindrical body.
